(19) United States
(12) Patent Application Publication
(10) Pub. No.: US 2013/0303971 A1
(43) Pub. Date: Nov. 14, 2013

(54) CATHETER AND SHUNT SYSTEM INCLUDING THE CATHETER

(71) Applicants: David Mortimer Budgett, Auckland (NZ); Ellyce Francis Stehlin, Auckland (NZ); Andrew James Taberner, Auckland (NZ); Poul Michael Fonss Nielsen, Auckland (NZ); Simon Charles Malpas, Auckland (NZ)

(72) Inventors: David Mortimer Budgett, Auckland (NZ); Ellyce Francis Stehlin, Auckland (NZ); Andrew James Taberner, Auckland (NZ); Poul Michael Fonss Nielsen, Auckland (NZ); Simon Charles Malpas, Auckland (NZ)

(73) Assignee: TELEMETRY RESEARCH LIMITED, Auckland (NZ)

(21) Appl. No.: 13/821,948

(22) PCT Filed: Sep. 9, 2011
(86) PCT No.: PCT/NZ2011/000185

§ 371 Date: Jun. 17, 2013

(30) Foreign Application Priority Data
Sep. 10, 2010 (NZ) 587910

(51) Int. Cl. A61M 27/00

(52) U.S. Cl. CPC A61M 27/006
USPC 604/9

(57) ABSTRACT

In one embodiment of the invention a catheter (203) comprises a body having at least one inlet aperture (21, 24), at least one outlet aperture (22, 25), and at least one passage (20, 23) between the at least one inlet aperture (21, 24) and the at least one outlet aperture (22, 25). The catheter (203) is provided with pumping means (32) for selectively pumping fluid from one of said apertures (21, 22, 24, 25) to another of said apertures (21, 22, 24, 25). Methods of operating such a catheter are also disclosed.

### FIELD OF INVENTION

The invention relates to catheters, and in particular, but not exclusively, to a long-term implantable catheter which has an active mechanism to reduce the incidence of the catheter becoming blocked, and a shunt system which includes the catheter.

### BACKGROUND

Hydrocephalus is one of the most common paediatric neurological disorders. The landmark feature of the disease is the accumulation of cerebrospinal fluid (CSF) in the ventricles of the brain causing their expansion. Blockages in the brain's ventricular system lead to accumulation of CSF and disruption of normal CSF circulation. When such blockages cannot be resolved an increase in intra-cranial pressure (ICP) occurs as the rate of CSF absorption into the bloodstream cannot match the ventricular system's production. Eventually, the increase in ICP causes the ventricles containing CSF to expand, which can lead to serious complications due to the displacement of brain tissue and compression of blood vessels.

The standard procedure for treating Hydrocephalus is to insert a shunt to drain excess fluid from the ventricles. Most commonly, the control of the fluid flow is achieved by a differential pressure valve allowing fluid to only flow when ICP is above the shunt's preset value. Fluid is typically shunted to the peritoneal space, with the right atrium of the heart and plural space also viable, but more complication prone, destinations. Shunts greatly improved the prognosis of the hydrocephalus patient; however they themselves are associated with a large number of complications. It is generally expected that 50% of shunts will have failed within 2 years of implantation. Despite new shunt technology, these failure rates have remained relatively steady since the development of the hydrocephalus shunt in the 1950s. Of all shunt failures, 70% are due to ventricular catheter occlusions (Drake, J. M., J. R. W. Kestle, and S. Tuli, CSF shunts 50 years on—past, present and future. Child's Nervous System, 2000. 16(10): p. 800-804.), (Kestle, J., et al., Long-term follow-up data from the Shunt Design Trial. Pediatr Neurosurg, 2000. 33(5): p. 230-236.). Diagnosis of failure requires expensive imaging techniques to observe ventricular size and short-term percutaneous ICP monitoring. Invasive surgery is required to remove and replace the blocked shunt. The cost of treating hydrocephalus in the US in the year 2000 was estimated at $1 billion, with shunt revisions responsible for approximately half this cost (Patwardhan, R. V. and A. Nanda, Implanted ventricular shunts in the United States: the billion-dollar-a-year cost of hydrocephalus treatment. Neurosurgery, 2005. 56(1): p. 139-44; discussion 144-5.).

Recent developments to decrease shunt failure rates include adjustable pressure valves, flow-regulating valves and anti siphoning devices. Adjustable pressure valves allow for the pressure threshold setting to be altered after implantation. Shunts fitted with such valves provide a solution to constant, consistent over or under drainage due to an incorrect pressure setting. However, overall the valves have not been found to significantly reduce failure rates. Some catheters of the prior art use flow limiting valves rather than a standard differential pressure valve.

The flow regulating shunt design was tested in a long-term shunt study by Kestle et al, along with new anti-siphoning devices. Anti-siphoning devices are specifically targeted to overcome the hydrostatic forces from the shunt's column of water which, when a patient changes posture, can cause severely negative ICP. Anti-siphoning devices work to counter-act the hydrostatic force by increasing resistance in the shunt line when ICP goes negative. The study revealed such new shunt designs have no advantage over standard valve designs. Flow regulating valves are often influenced by simple movements and anti-siphoning devices are highly vulnerable to changes in external pressure. Most significantly, the anti-siphon devices only function short-term until scarring interferes and prevents their function (Aschoff, A., et al., Overdrainage and shunt technology. Child's Nervous System, 1995. 11(4): p. 193-202.).

Shunt technology focusing on overcoming catheter occlusions have also been developed. U.S. Pat. No. 5,584,314 describes a self cleaning inlet head which works in line with the shunt at the proximal end. The device involves a moving piston inside the catheter working to dislodge particles in combination with a hydraulic mechanism. A self-cleaning medical catheter has also been described which uses vibration of a proximal orifice of the catheter to dislodge clogging deposits (U.S. Pat. No. 4,698,058). Additional mechanically active catheters include a drug-delivery catheter which uses piezoresistive activity to dislodge crystallised drugs (U.S. Pat. No. 4,509,947) and a drug-delivery catheter which uses ultrasonic vibrations to enhance localised drug distribution (U.S. Pat. No. 5,767,811).

Additional patents have been issued to focus on distal catheter occlusions, more common in the adult hydrocephalus population. These include devices for anchoring implanted catheters in a specific location and orientation such as U.S. Pat. Nos. 6,554,802 and 6,562,005.

The reference to any prior art in the specification is not, and should not be taken as, an acknowledgement or any form of suggestion that the prior art forms part of the common general knowledge in any country.

It is the object of the present invention to provide a catheter and/or a shunt system including the catheter which can maintain the opening of the catheter for periods of time which are longer than are currently achieved using passive catheters, or to at least provide the public with a useful choice.

Other objects of the present invention may become apparent from the following description, which is given by way of example only.

### SUMMARY OF INVENTION

According to one aspect of the present invention there is provided a catheter comprising a body having at least one inlet aperture, at least one outlet aperture, and a passage between the at least one inlet aperture and the at least one outlet aperture, the catheter provided with pumping means for selectively pumping fluid from one of said apertures to another of said apertures.

Preferably the catheter is provided with a plurality of inlet apertures.

Preferably the catheter is provided with a plurality of outlet apertures.

Preferably the pumping means is operable to pump fluid from any one of said apertures to any other one of said apertures.

Preferably the pumping means comprises at least one actuator operable to compress a resiliently flexible portion of the body of the catheter.

Preferably the resiliently flexible portion comprises a biocompatible medical grade silicone elastomer.

Preferably the at least one actuator is operable to dilate the resiliently flexible portion of the body.

Preferably the pumping means is operable to create a negative pressure in the passage.

Preferably the pumping means comprises a plurality of said actuators, each said actuator associated with a respective resiliently flexible portion.

Preferably the actuators are linear actuators.

Preferably the actuators comprise piezo electric actuators.

Preferably the actuators are embedded within a housing.

Preferably the pumping means comprises a rotor.

Preferably the pumping means comprises a cam.

Preferably the pumping means is operable as a peristaltic pump.

Preferably the pumping means is adapted to provide a required resistance to fluid flow through the passage when in a non-powered state.

Preferably the entire body is made from a resiliently flexible material.

Preferably the catheter comprises a control means for controlling the pumping means.

Preferably the control means comprises a microprocessor.

Preferably, in use, the control means operates the pumping means at a substantially constant speed.

Preferably, in use, the control means operates the pumping means to provide a substantially constant flow rate.

Preferably the catheter comprises a pressure sensor.

Preferably the pressure sensor is positioned to allow measurement of intra-cranial pressure (ICP) when in use.

Preferably, in use, the control means receives a signal from the pressure sensor.

Preferably, in use, the control means operates the pumping means to increase flow through the catheter if the ICP is greater than a predetermined pressure.

Preferably, in use, the control means operates the pumping means to decrease or halt fluid flow through the catheter if the ICP is lower than a predetermined pressure.

Preferably the catheter further comprises an electrically actuable portion associated with at least one of the inlet aperture and outlet aperture which is adapted to reversibly deform the respective aperture when actuated.

According to a second aspect of the present invention there is provided a catheter comprising a body having an inlet aperture, an outlet aperture, and a passage between the inlet and outlet apertures, the catheter further comprising an electrically actuable portion associated with at least one of the inlet aperture and outlet aperture which is adapted to reversibly deform the respective aperture when actuated.

Preferably the electrically actuable portion substantially surrounds the aperture.

Preferably the electrically actuable portion is formed integrally with the body.

Preferably the electrically active portion is formed from a separate material to the body.

Preferably the electrically actuable portion comprises an electro-active polymer.

Preferably the electrically actuable portion comprises a memory shape alloy or micro electromechanical system (MEMS) actuators.

Preferably the actuable portion is compliant.

Preferably the actuable portion is formed from a biocompatible medical grade silicone elastomer.

Preferably the catheter comprises a flow control valve adapted to control fluid flow between the inlet and the outlet.

According to a third aspect of the present invention there is provided a catheter comprising a body having an inlet aperture, an outlet aperture, and a first passage between the inlet and outlet apertures, the catheter further comprising a second passage which intersects the first passage proximate the inlet aperture, the apparatus further comprising an electrically actuable portion adapted to displace fluid from the second passage into the first passage.

Preferably the second passage comprises a reservoir portion.

Preferably the electrically actuable portion is operable to decrease an internal volume of the reservoir portion.

Preferably the electrically actuable portion comprises an electro-active polymer.

Preferably the electrically actuable portion comprises a memory shape alloy or micro electromechanical system (MEMS) actuators.

Preferably the catheter comprises a flow control valve adapted to control fluid flow between the inlet and the outlet.

According to a further aspect of the present invention there is provided an implantable shunt system comprising the catheter of any one of the first, second or third aspects.

Preferably the system further comprises a power source.

Preferably the power source comprises a battery.

Preferably the power storage means comprises a capacitor, preferably a super capacitor.

Preferably the system comprises an inductive power transfer pickup.

Preferably the system comprises an accelerometer adapted to sense the orientation of the system.

Preferably the system comprises telemetry means for transmitting information from a sensor associated with the catheter.

Preferably the system comprises an external monitor/controller.

Preferably the monitor/controller sends information by telemetry to the catheter.

Preferably the monitor/controller receives information by telemetry on the status of the catheter.

Preferably the monitor/controller provides the inductive power source to activate and energise the implantable shunt system.

Preferably the monitor/controller contains an atmospheric reference pressure sensor.

Preferably the monitor/controller includes an algorithm to convert data received by sensor(s) in the implantable shunt system to instructions for the patient.

Preferably the monitor/controller incorporates a graphical user interface to display instructions and information on the status of the shunt system to the patient.

Preferably the monitor/controller incorporates on-board memory to store data received from the shunt system and the means of uploading data to a remote computer.

According to a further aspect of the present invention there is provided a catheter substantially as herein described with reference to the accompanying drawings.

According to a further aspect of the present invention there is provided an implantable shunt system capable of controlling fluid flow, the system including:
a valve system and/or pumping means capable of regulating fluid flow;
a sensor to detect the need to operate the valve;
a power source to enable the active components to be energised; and
a telemetry system to allow the active components to be controlled and interrogated.

According to a still further aspect of the present invention there is provided a catheter comprising a body with a proximal aperture, a distal aperture and an internal passage connecting the proximal and distal apertures, a pressure sensor capable of measuring pressure within the passage, a controller, and means for selectively bringing the pressure sensor into fluid communication with the distal aperture while isolating the pressure sensor from the proximal aperture, wherein the controller determines a reference pressure for the pressure sensor by isolating the proximal aperture, bringing the pressure sensor into fluid communication with the distal aperture and measuring the pressure in the passage.

According to a still further aspect of the present invention there is provided a method of operating a catheter comprising controlling if a pressure sensor associated with the catheter is in fluid communication with a fluid in a user's brain or with a fluid in another part of a user's body, bringing the pressure sensor into fluid communication with the fluid which is in the other part of the body, computing a reference level, determining whether an ICP is elevated or depressed by measuring ICP with the pressure sensor, and taking an appropriate action based on whether the ICP is elevated or depressed.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE FIGURES

FIG. 1 Is a diagrammatic side view of a shunt system according to one embodiment of the present invention in situ in a patient.

FIG. 2 is a diagrammatic cross-section side view of an inlet portion of a catheter according to one embodiment of the present invention.

FIG. 3 is a diagrammatic cross-section side view of a catheter according to a second embodiment of the present invention.

FIG. 4 is a diagrammatic cross-section side view of the inlet portion of the catheter of FIG. 3.

FIG. 5 Is a diagrammatic side view of a shunt system according to another embodiment in situ in a patient.

FIG. 6 is a diagrammatic side view of a cam and cam motor of a further embodiment of a catheter of the present invention.

FIG. 7 is a diagrammatic side view of a catheter with a pumping means.

FIG. 8 is a flow chart of a basic algorithm for monitoring the performance of a catheter which is provided with a pumping means.

FIG. 9 is a diagrammatic side view of a catheter which is suitable for use with the algorithm of FIG. 8.

FIG. 10 is a flow chart of a basic algorithm for periodically back-flushing the inlet apertures of a catheter.

FIG. 11 is a diagrammatic side view of a catheter which supports calibrating a pressure sensor.

FIG. 12 is a flow chart of a basic algorithm for updating a pressure reference level and controlling a pressure based on that reference level.

### BRIEF DESCRIPTION OF EMBODIMENTS OF THE INVENTION

The present invention pertains to catheters and shunt systems with the ability to avoid or remove proximal occlusions.

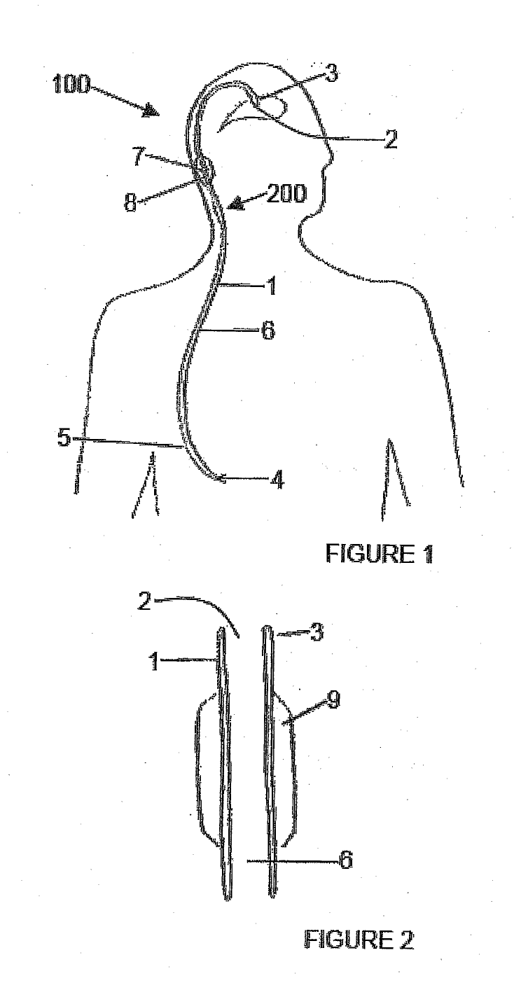

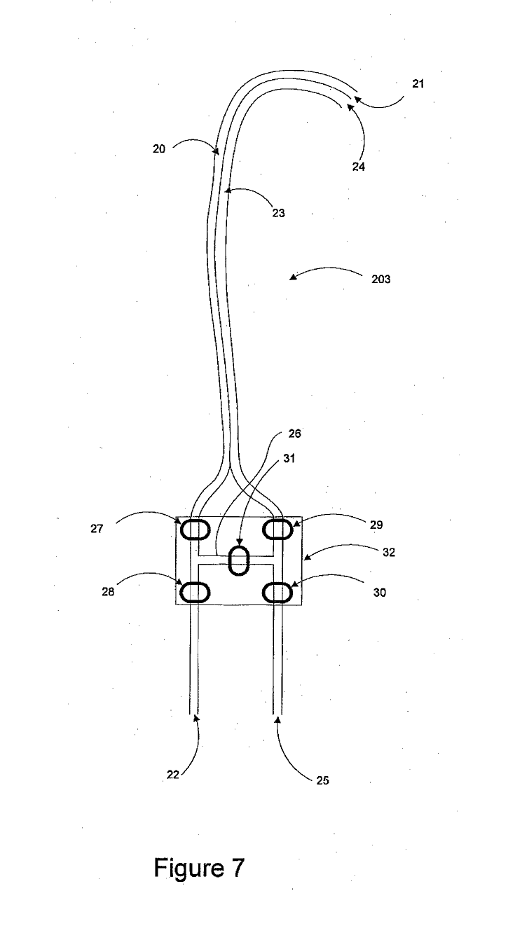

Referring first to FIGS. 1 and 2, one embodiment of the invention is described with relation to application in the hydrocephalus shunt system, generally referenced by arrow 100. The shunt system 100 includes a catheter 200 comprising a body 1, for example a tube made from a suitable polymer, which has an inlet aperture 2 at the proximal end 3 which sits inside the ventricle of the brain to drain fluid to an outlet aperture 4 provided at the distal end 5 via a passage 6 provided in the body 1. The outlet aperture 4 is typically located in the abdomen. Control of fluid through the catheter 200 may be achieved by a control valve 7 which is contained inside a valve housing 8, which defines the separation of the proximal and distal catheters. Alternatively the control may be achieved through the use of one or more actuators, as described further below with reference to FIGS. 7, 9 and 11.

The valve housing may be made from a biocompatible material such as titanium or ceramics.

Referring in particular to FIG. 2, in one embodiment, the proximal end 3 of the catheter 200 has the ability to deform and change the profile of the inlet aperture 2 for the purpose of dislodging occluding tissue. In this embodiment, profile changes are achieved by placement of electrically actuated material 9 around the proximal end 3 of the catheter 200. Repeated profile changes may be achieved by using materials which change shape when supplied with electrical stimulation. Alternatively electro-active polymers may be used in the material of the proximal tip which, when electrically activated, contract to change the opening profile of the inlet aperture 2. The electrically controlled deformations preferably allow for relatively small, controlled, changes in the inlet aperture 2 dimensions, allowing the standard circular tip profile to repeatedly cycle from a circular profile to an ellipse. Additionally or alternatively the catheter 200 may have the ability to deform and change the profile of the outlet aperture 4 in the same manner.

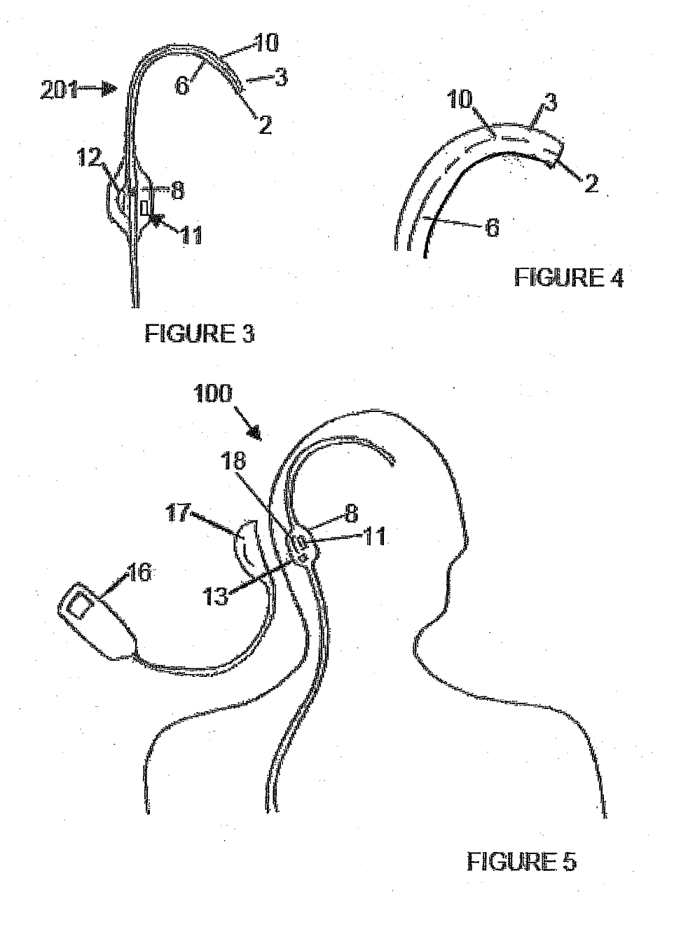

Referring next to FIGS. 3 and 4, another embodiment of a catheter according to the invention is generally referenced by arrow 201. In this embodiment a dual proximal catheter tube is used, that is, the catheter 201 is provided with a second passage 10 in addition to the first passage 6. The second passage 10 intersects the first passage 6 near the inlet 2, as is best seen in FIG. 4.

The secondary passage 10 is used to divert and store fluid from the main passage 6. The stored fluid can then be displaced in an opposing direction to the drainage fluid in the first passage 6. This alternate direction of flow prevents and removes blockages by periodically forcing occluding material in an opposing direction to normal fluid drainage.

A controller 11 for the secondary passage 10 may be contained in the catheter valve housing 8, close to the valve 7 which controls overall flow through the main passage 6 to the outlet 4. In one embodiment, the secondary passage 10 defines a reservoir 12 at its terminating end, which in one embodiment may be just proximal to the valve 7. Activation of the self flushing catheter system is achieved by compressing the reservoir 12, thereby forcing fluid towards the inlet aperture 2. In other embodiments (not shown) the second passage 10 may contain sufficient fluid that a distinct reservoir portion is not required. In such embodiments the second passage 10, or a portion of the second passage 10, may be deformed or compressed in order to displace the fluid therein back towards the inlet 2. This compression can be achieved using electrically actuated materials, as is described above with reference to the inlet 2, or through the use of one or more actuators, as described further below with reference to FIGS. 7, 9 and 11. The controller 11 may be microprocessor based, as will be apparent to those skilled in the art.

The intersection between the first passage 6 and the second passage 10 also allows for refilling of the reservoir 12 with fluid from the inlet 2 and primary passage 6.

In another embodiment (not shown), the second passage 10 may have a second inlet which is independent from inlet 2.

In some embodiments the system 100 includes a miniature pressure sensor (not shown) that monitors pressure at the proximal end 3 of the catheter 200, 201. For example, in systems intended for use with hydrocephalus patients the sensor is positioned on the catheter such that it sits in the ventricle for a measure of true intracranial pressure. The pressure sensor data may be used as an indication of the effectiveness of the catheter 200, 201, allowing for an instant indication of the shunt's ability to control ICP in a hydrocephalus patient. Alternative locations for the pressure sensors include any of the shunt-valve housing 8 and distal end of the catheter 5.

Referring next to FIG. 5, in one embodiment an accelerometer 13 is included in the active catheter system 100. The accelerometer may be used to monitor changes in the patient's posture.

Using the accelerometer 13 in conjunction with the pressure sensor, specific changes in intra-cranial pressure due to posture changes can be monitored, providing early evidence of any persisting occlusions. The accelerometer 13 is not required to sit in the catheter itself. In the embodiment shown in FIG. 5 the accelerometer 13 is located within the shunt-valve housing 8, which sits outside of the patient's skull.

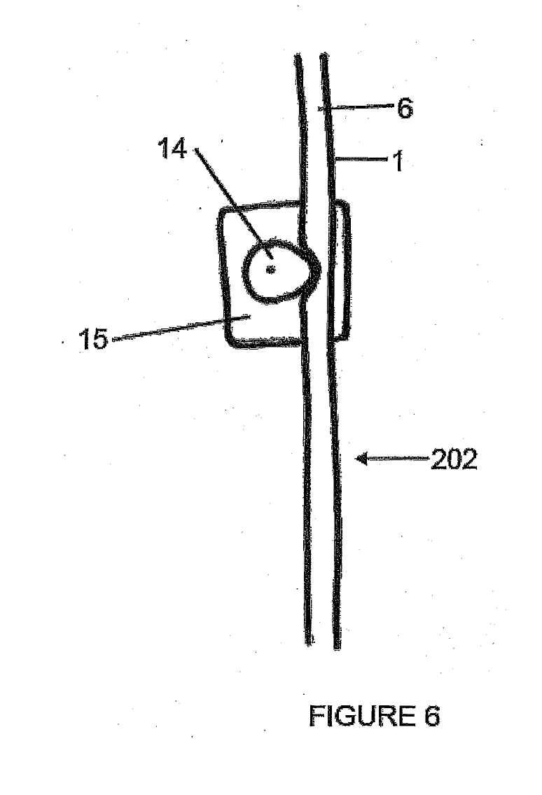

Referring next to FIG. 6, in order to overcome the siphoning effect due to posture changes in the hydrocephalic patient application, in one embodiment a catheter, generally referenced by arrow 102, includes a posture dependent shunt resistance component, for example a cam 14, which may replace the main control valve. Accelerometer data allows the siphon effect to be forewarned and compensated for. This compensation is achieved by activating a cam motor 15 to rotate the cam 14 into a position which compresses a flexible portion of the catheter tube 1, thereby causing a change in shunt line resistance. The degree of compression is variable and reversible based on the accelerometer sensing a patient changing posture and acts to prevent the occurrence of an extreme negative intra-cranial pressure. In this embodiment the accelerometer may be used in closed loop control of the additional posture dependent resistance component.

In some embodiments, both shunt line resistance and occlusion prevention and debris removal will be achieved using the same mechanism. A shunt system provided with a catheter 202 as shown in FIG. 6 may use the cam 14 to promote fluid flow though the passage 6, in the manner of a peristaltic pump. The rotational speed of the cam 14 may be controlled to regulate flow rate. This technique may be used in place of the standard control valve 7. When an accelerometer senses a posture change in the patient from horizontal to upright, the cam 14 is activated to provide an increased resistance to flow, preventing the siphoning effect. The cam 14 also has the ability to be activated in a reverse flow mode to flush occlusion causing debris out of the catheter. When the cam 14 is not being powered or rotated, it may be set to provide a set resistance to flow, in a comparable fashion to standard valves 7.

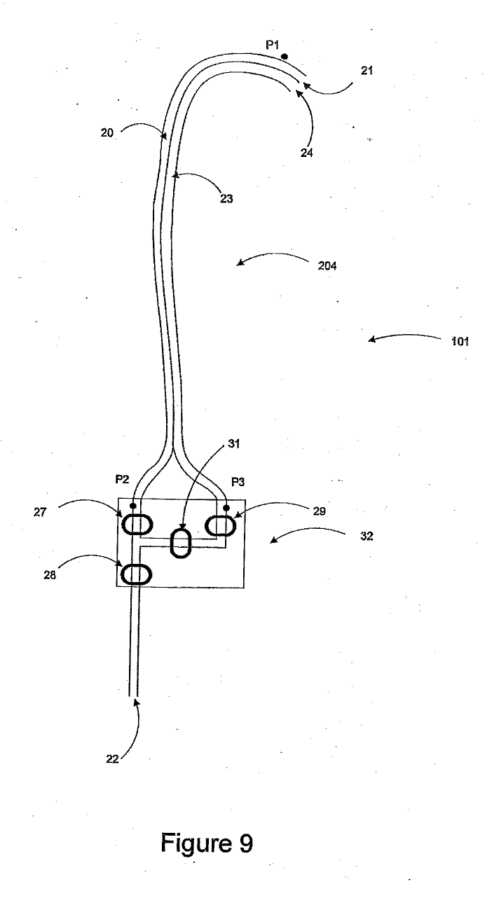

In a variation of the catheter shown in FIG. 6, in some embodiments (not shown) a catheter may have a body which is provided with two parallel passages, each with its own separate inlet and outlet. A cam and cam motor may be provided for each passage. In this way one of the cams may be operated in a reverse flow flushing mode while the other operates in a normal mode. This may assist in keeping the pressure in the ventricle constant even while one passage is being flushed. In other embodiments a system may include two separate catheters 200, 201, 202 with a common control means 11. In other embodiments, for example the system shown in FIG. 9, a system may include two inlets with passages which converge to a single outlet. In this configuration the fluid can be circulated through the proximal catheters to flush the inlet without causing substantial changes in the intercranial pressure.

Referring back to FIG. 5, the active catheter system 100 may be powered wirelessly by transcutaneous energy transfer. This may be achieved with inductive power transfer, for example from an external hand held power transfer means 16 which contains a primary coil 17 for setting up a magnetic field that induces a current in a secondary coil 18 which is contained in the electronics of the catheter controller 11. The power transfer means may also function as a monitor and/or controller, and may send and/or receive information by wireless telemetry, for example information on the status of the implantable shunt system. It may also be provided with on-board memory to store data received from the shunt system, and may have a means of uploading data to a remote computer.

The monitor/controller may include an algorithm to convert data received by sensor(s) in the implantable shunt system to instructions for the patient. A graphical user interface may be provided to display instructions and information on the status of the shunt system to the patient. The monitor/controller may contain an atmospheric reference pressure sensor.

In one embodiment the system 100 is only active when the external magnetic field producing power transfer means 16 is active and in range. The system 100 is therefore battery free and both the active occlusion resisting action and sensing system will cease activity when the external powering wand is removed. In another embodiment a super capacitor (not shown) can be used to allow for periodic activity of the active catheter. The super capacitor can be charged with holding the power transfer means 16 over the catheter control unit 11. When the power transfer means 16 is removed, the catheter remains active for some time.

Alternatively, the system includes a rechargeable battery (not shown) allowing continual activity when the external magnetic field supply is not applied. The battery supplies power to the device when the external wand is not being held over the system and continual monitoring of patient and catheter condition is realised. The battery is recharged when the external supply is in range.

In one embodiment telemetry is used to transmit data from the implanted active catheter's pressure sensor, accelerometer and/or other sensor(s). The information allows for improvements in the control of an individual patient's catheter activity and for quick diagnosis of problems in the active catheter system. Pressure and accelerometer data therefore have the ability to be used in closed control within the shunt system 100 itself or, alternatively the information is transmitted out of the active catheter system for external interpretation and use. The telemetry data may be received wirelessly by power transfer means 16.

Excluding the deformable proximal tip of the catheter, the bulk of the electronics are preferably contained in an encapsulated unit surrounding the catheter. In the hydrocephalus application, this unit may be situated alongside the shunt valve housing 8, or integrated into the valve housing 8. This allows the electronics to be located outside of the skull, providing the opportunity for close contact between inductively coupled coils 17, 18 for power transfer and wireless communication pickup.

Referring next to FIG. 7 a catheter provided with an active pumping means is generally referenced by arrow 203. The catheter 203 is provided with a first passage 20 having a proximal aperture 21 and a distal aperture 22. The catheter is further provided with a second passage 23 having a proximal aperture 24 and a distal aperture 25.

A third passage 26 connects the first and second passages 20, 23.

The catheter is provided with a first actuator 27 between the proximal aperture 21 and the third passage 26, a second actuator 28 between the third passage 26 and the distal aperture 22, a third actuator 29 between the proximal aperture 24 and the third passage 26, and a fourth actuator 30 between the third passage 26 and the distal aperture 25. The third passage 26 is provided with a fifth actuator 31. Each actuator is connected to an adjacent resiliently deformable portion of the body.

In a preferred embodiment the electrical connection between the actuators and a suitable power source and/or control mechanism may be embedded into the catheter body.

Each of the actuators 27-31 is capable of compressing the adjacent portion of the body, and thereby restricting flow through the passage with which the actuator is associated. The compression of the body, and passage, has the effect of decreasing the internal volume of the passage.

The connection between the actuators 27-31 and the body is also such that the actuators can dilate the passage within the body, thereby increasing the internal volume of the passage, and thereby creating a negative pressure within the passage.

By correctly timing the opening and closing of the actuators 27-31, a pumping action can be achieved to draw fluid from any one of the apertures 21, 22, 24, 25 and deliver fluid to any aperture. Thus, the actuators 27-31, together with the portion of the passages on which the actuators act, define a pumping means, generally referenced by arrow 32.

In a normal or routine state, the pumping means draws fluid from either aperture 21 or 24 and delivers the fluid to either aperture 22 or 25 for the purpose of lowering ICP. However, it is also possible to direct flow in different directions to enable flushing of any aperture 21, 22, 24, 25, or any passage connecting an aperture to the pump 32.

By way of example, the operation of the pumping means 32 to pump fluid from aperture 21 to aperture 22 is described below.

The initial position of the pumping means 32 has actuator 28 in an open position, and all other actuators closed. The first step is to close the destination actuator 28. Next, the source actuator 27 is opened. Next, the crossover actuator 31 is opened, thereby drawing fluid through the source aperture 27.

The next step is to close the source actuator 27. Next, the destination actuator 28 is opened. Finally, the crossover actuator 31 is closed, expelling fluid through the destination aperture 28. This returns the system to the starting position, and another cycle may be initiated if required.

The pumping means 32 described above is preferably implemented with individually controlled actuators such as the SQL-RV-1.8 linear piezo electric motion control system available from New Scale Technologies. This motor has an I2C interface (also referred to as a “two-wire” interface) allowing direct connection to a nRF24LE1 “system on” chip, available from Nordic Semiconductor, which contains an 8051 microprocessor and 2.4 GHz radio transceiver.

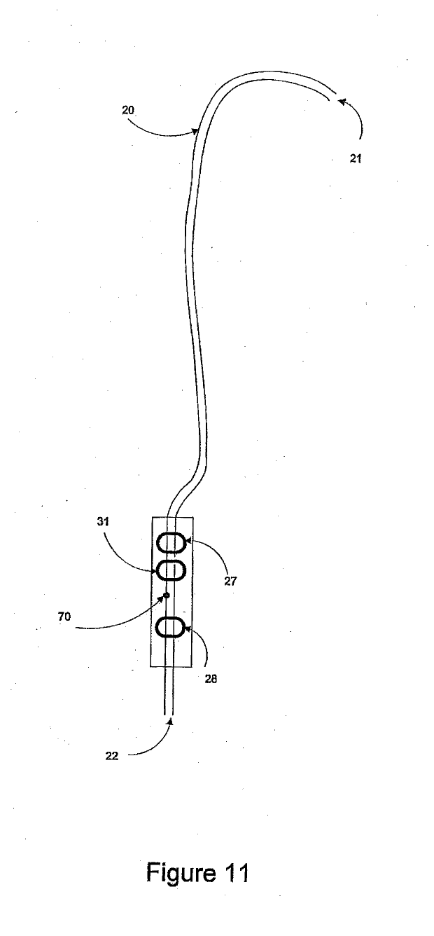

In another embodiment, shown in FIG. 11, a pumping means may comprise three or more of said actuators in a row, the actuators being actuable in a repeating sequence one after the other to provided a peristaltic pump action on the passage.

In a further embodiment (not shown), a pumping means may comprise a single linear actuator, such as a SQL-RV-1.8 linear piezo electric motion control system, which operates a cam, for example via a rhombic drive. The cam may implement the peristaltic pumping action.

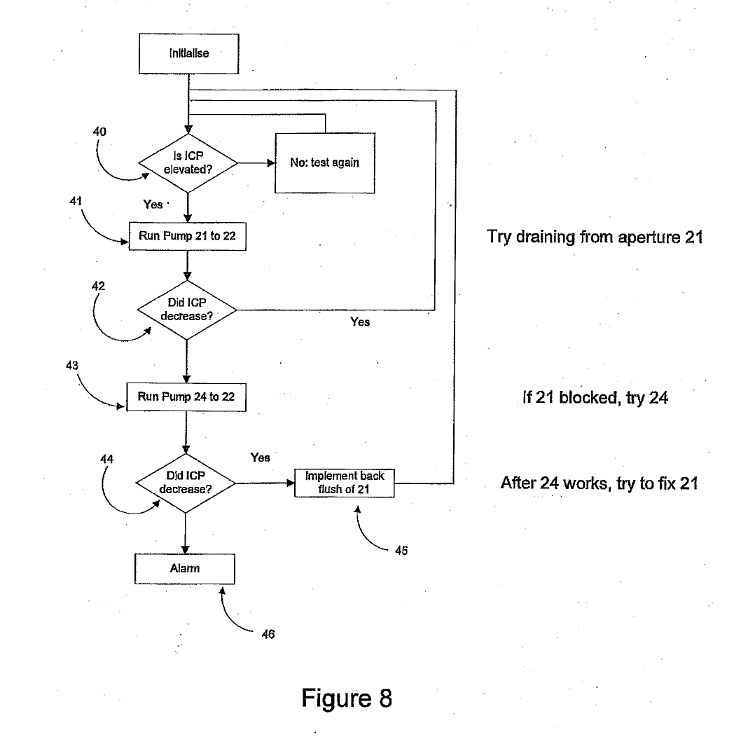

Referring next to FIG. 8, an algorithm is provided for controlling a catheter 204 which is part of a shunt system 101 of the present invention. The shunt system 101 is illustrated in FIG. 9. The catheter 204 is similar to the catheter 203 shown in FIG. 7, and similar reference numerals are used to refer to similar features.

The system control means (not shown) may use the algorithm to monitor ICP and reduce ICP pressure if it is too high. In a preferred embodiment the algorithm is implemented in a microprocessor.

The shunt system 101 comprises two proximal apertures 21, 24, which in use are located in the ventricle of the brain (not shown). A third aperture 22 is located at the distal end of the catheter.

At step 40 the ICP is measured by pressure sensor P1 and the system checks whether it exceeds a predetermined threshold pressure. If the ICP is elevated, a pumping means 32 is activated at step 41 to pump fluid from aperture 21 to aperture 22. The pumping means 32 is operated for a fixed duration and then stopped.

At step 42 the ICP is again measured. If it has dropped, the process loops back to step 40. If the ICP pressure does not drop after a pumping action, then at step 43 the system attempts to pump fluid from the second aperture 24. The ICP is again measured at step 44. If pumping from aperture 24 to aperture 22 is successful, as determined by a drop in the ICP, then it is assumed that aperture 21 is blocked and an attempt to remove the blockage in aperture 21 by back flushing fluid from aperture 22 or 24 is initiated at step 45, after which the process returns to step 40. If the ICP has not decreased at step 44, then it is concluded that the ICP cannot be managed, and an alarm is raised at step 46.

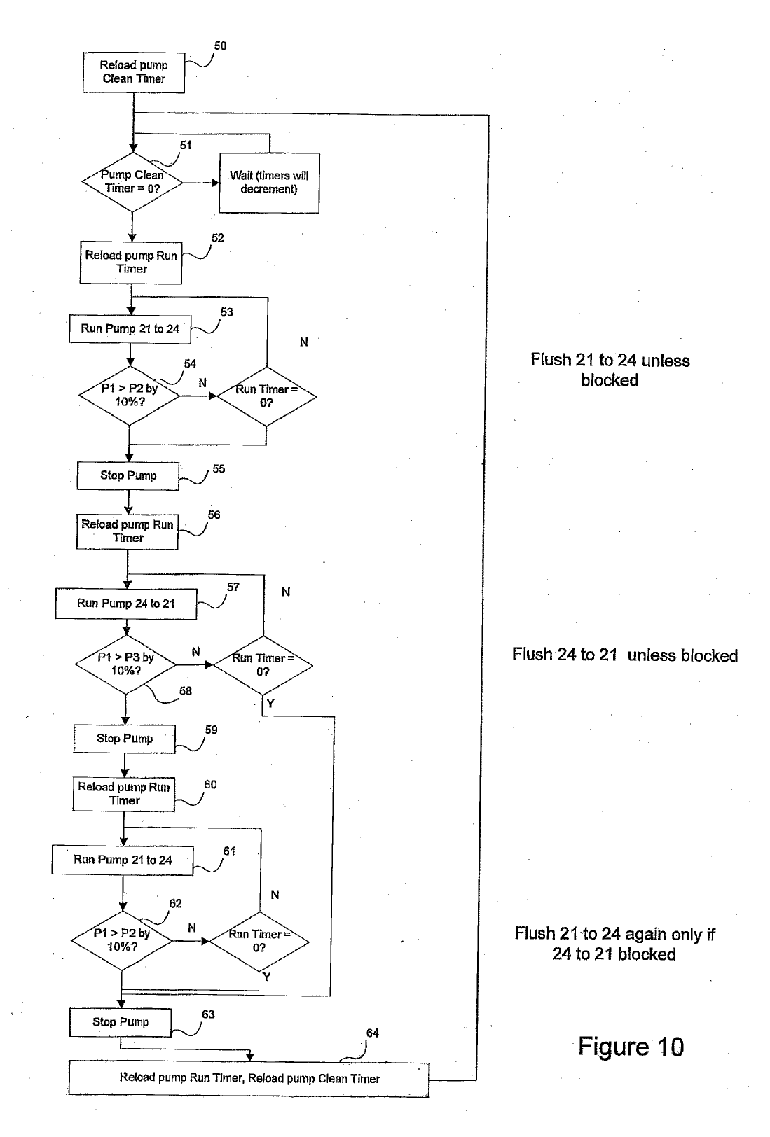

More detail of an algorithm for back flushing is described below with reference to FIG. 10.

The algorithm shown in FIG. 8 is very simple and is provided for the purpose of illustrating the improved robustness of the invention to a blockage of a proximal aperture 21, 24. Those skilled in the art will appreciate that the algorithm can be expanded to allow the symmetrical use of aperture 21 and aperture 24; to allow aperture 24 to be used to return ICP to normal levels before implementing the back flush procedure to attempt to clear aperture 21; and to enable the detection of a blockage in aperture 24 and provide a corrective action. The algorithm is also expandable to accommodate periodic retries of corrective action and produce multiple alarms, and to provide diagnostic information including using a telemetry system to report on parameters such as attempts made, pressures measured, power status and radio performance.

Referring next to FIGS. 9 and 10, an algorithm, implemented in a microprocessor, for performing an automated black flushing process to prevent debris from blocking an aperture of the shunt system 101 is described.

The shunt system has two inlet apertures 21, 24. A pressure sensor P1 is located in the ventricle. A second pressure sensor P2 is located inside passage 20 running from aperture 21 to the pumping means 32. A third pressure sensor P3 is located in the second passage 23 running from aperture 24 to the pumping means 32.

The microprocessor implements two timers that decrement on a periodic basis, based on regular interrupts. The first timer is called the Pump Clean Timer. The Clean Pump Timer is reset at step 50. At step 51 the current value of the timer is monitored. When the Pump Clean

Timer counts down to zero, the process of pumping fluid from one of apertures 21, 24 to aperture 22 is interrupted, and fluid is pumped from aperture 21 to aperture 24, or from aperture 24 to aperture 21. Thus, the algorithm of FIG. 9 is used for periodically causing a flow out of each aperture 21, 24 in turn to push any debris that may have entered the inlet back into the ventricles.

The flushing process starts at step 52 by resetting the Pump Run Timer. At step 53 the pumping means 32 is run, with fluid entering aperture 21 and being pumped out of aperture 24. At step 54 the pressure measured in the ventricles by pressure sensor P1 is compared to the pressure measured in passage 20 by pressure sensor P2. If the pressure at P1 is greater than that at P2 by more than a threshold margin, for example 10%, then a blockage is indicated. If a blockage is detected, the pumping means 32 is stopped at step 55 and then reversed. Otherwise, the pumping means continues to run until the Pump Run Timer reaches zero.

At step 56 the Pump Run Timer is reloaded. The pumping means 32 is then run to pump fluid from aperture 24 to aperture 21 at step 57. This process normally continues for a fixed time as determined by the number loaded into the Pump Run Timer, after which the entire process resets.

The reverse pumping action continues for a period defined by the Pump Run Timer unless a blockage is indicated by the pressure in passage 23 (measured by pressure sensor P3) exceeding P1 by more than a threshold margin, for example 10%, as shown at step 58. If a blockage condition is indicated, then at step 59 the pumping means 32 is stopped and at step 60 Pump Run Timer is again reset. At step 61 the pump direction is again reversed to attempt to remove the cause of the blockage.

If a blockage is again detected at step 62 then an alarm may be raised (not shown), or the system may make a note of the continued blockage and may raise an alarm if the blockage is still present after a predetermined number of unsuccessful cleaning cycles have been attempted.

After these processes are complete the pumping means is stopped at step 63. At step 64 the Pump Clean Timer is reloaded. This configures the delay before the next clean cycle begins.

By providing the catheter with a pumping means which can produce a negative pressure inside a catheter that is occluded, it is possible to clear a blockage by drawing the obstruction through the catheter.

Pressure sensors can be prone to long term drift where, over an extended period of time, the value they report differs from the actual pressure they experience. The embodiment shown in FIG. 11 has a single pressure sensor, 70, which can be connected hydrodynamically to either the proximal aperture 21, or alternatively the distal aperture 22. By closing actuator 27 and opening actuator 28, the sensor 70 is connected to the distal aperture 22, and disconnected from the proximal aperture 21. Pressure measurements taken in this configuration are independent from the ICP pressure, and so may be used to derive a Reference Level against which an elevated ICP can be measured.

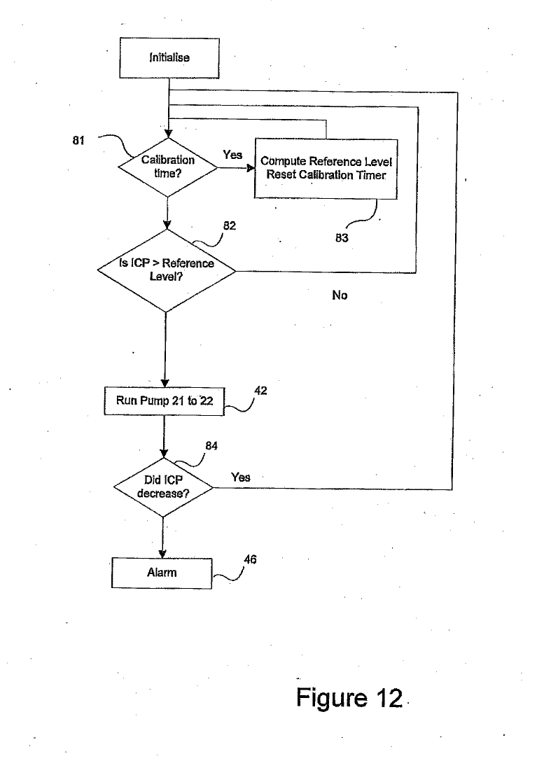

An algorithm showing how the Reference Level is used for the purpose of managing hydrocephalus is shown in FIG. 12. At step 81 the decision is made if a new Reference Level should be computed. This may be initiated in response to a command from the hand held controller 16, or after a timer interval, for example once per year. If a new Reference Level is required, then step 83 is implemented to acquire pressure data from the source unrelated to ICP. Once a valid Reference Level is obtained, step 82 will compare an ICP measurement against the Reference Level. If the ICP is elevated with respect to the Reference Level, a pumping action is performed by step 42. The simple pumping actions shown in FIG. 12 can be substituted by a more comprehensive response described in algorithms of FIG. 8 and FIG. 10 in conjunction with more versatile hardware shown in FIG. 7 and FIG. 9.

The derivation of the Reference Level may rely on recorded pressures from the distal aperture over a series of time intervals. This may be necessary to reduce artifacts that cause variation in the pressure at the location of the distal aperture. One example of the derivation is based on computing the mean pressure over a series of time intervals to use as a Reference Level. Another example of the derivation is to record pressure values over a 24 hour period and find the silent interval where the pressure variation is less than 1 mm Hg. The mean pressure value calculated over samples during the longest silent interval occurring within the 24 hour period may then be then taken as the Reference Level. Another example of the derivation is to record the Reference Level in memory, and only allow it to be updated if the newly computed Reference Level is within a fixed margin from the existing Reference Level. An example of the fixed margin is 0.5 mm Hg.

The shunt system shown in FIG. 11 also supports self-calibration of the pressure sensor span. This test is achieved by first closing actuator 28, opening actuators 27 and 31. Next, actuator 27 is closed. This encloses a known volume of fluid within the portion of the passage containing the pressure sensor 70. Closing actuator 31 will generate a known pressure increase to enable the span of pressure sensor 70 to be calibrated.

The hydrocephalus algorithm shown in FIG. 12 is for the purpose of illustrating the process of making use of a reference level, and from time to time, updating the reference level. Those skilled in the art would appreciate that the method may be used in any embodiment of the invention which has the capability of measuring the pressure at the distal aperture independently from the pressure of the proximal aperture. The microprocessor is also capable of implementing more comprehensive processing to manage the flow of fluid based on all sensory information available and in combination with other algorithms already described, in addition to implementing diagnostic, maintenance and telemetry functions.

In some embodiments the pumping means of the shunt system may be positioned towards the distal end of the catheter, for example in the abdomen of the patient, allowing the proximal end to remain undisturbed if a revision is required.

In a preferred embodiment the system includes a pressure sensor signal conditioning arrangement which provides an analogue signal to an analog to digital converter located in the nRF24LE microprocessor. An Inductive Power section receives power from a magnetic field and maintains battery charge. Motors provide control and feedback to the actuators and communicate with the nRF24LE using the I2C serial protocol. The nRF24LE1 interfaces to a 50 ohm 2.4 GHZ antennae.

Use of the active catheter of the present invention is not limited to shunted hydrocephalus patients. It may also be used in a drug delivery system, where it is also advantageous to prolong the life of the catheter from occlusion failure.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

Where in the foregoing description, reference has been made to specific components or integers of the invention having known equivalents, then such equivalents are herein incorporated as if individually set forth.

Although this invention has been described by way of example and with reference to possible embodiments thereof, it is to be understood that modifications or improvements may be made thereto without departing from the spirit or scope of the invention.

1. A catheter comprising a body containing a pumping means and having at least one inlet aperture, at least one outlet aperture, and at least one passage between the at least one inlet aperture and the pumping means, and at least one passage between the at least one outlet aperture and the pumping means, for selectively and reversibly pumping fluid from one of said apertures to another of said apertures.

2. The catheter of claim 1 wherein the catheter is provided with a plurality of inlet apertures independently connected to the pumping means.

3. The catheter of claim 2 wherein the catheter is provided with a plurality of outlet apertures independently connected to the pumping means.

4. The catheter of claim 1 wherein the pumping means is operable to pump fluid from anyone of said apertures to any other one of said apertures.

5. The catheter of claim 4 wherein the catheter comprises a controller which operates the pumping means to flush an aperture on a periodic basis.

6. The catheter of claim 5 wherein the controller operates the pumping means to unblock any aperture by reversing the direction of fluid flow.

7. The catheter of claim 5 wherein the-pumping means does not alter the pressure at any aperture outside of normal physiological levels.

8. The catheter of claim 1 wherein the pumping means comprises at least one actuator operable to compress or dilate a resiliently flexible portion of the body of the catheter.

9. The catheter of claim 8 wherein the pumping means is operable to create a negative pressure in the passage compared to a pressure at any aperture.

10. The catheter claim-8 wherein the pumping means comprises a plurality of said actuators, each said actuator associated with a respective resiliently flexible portion.

11. The catheter of claim-1 wherein the pumping means is adapted to provide a required resistance to fluid flow through the passage when in a non-powered state.

12. The catheter of claim-1 wherein a pressure sensor is positioned inside the pumping means to allow selective measurement of pressure from sections of the catheter leading to specific apertures.

13. The catheter of claim 12 wherein the pressure sensor is calibrated by measurement from one aperture of known pressure and then an unknown pressure is derived from the measurement from another aperture.

14. The catheter of claim 12 wherein the catheter comprises a controller which controls the pumping action to flush an aperture based on feedback from the pressure sensor.

15. A catheter comprising a body having an inlet aperture, an outlet aperture, and a passage between the inlet and outlet apertures, the catheter further comprising an electrically actuable portion associated with at least one of the inlet aperture and outlet aperture which is adapted to reversibly deform the respective aperture when actuated.

16. The catheter of claim 14 wherein the electrically actuated portion can switch reversibly between a circular profile to an elliptical profile without a significant change in fluid flow.

17. The catheter of anyone of claim 14 wherein the electrically actuable portion substantially surrounds the aperture.

18. The catheter of claim 14 wherein the electrically actuable portion comprises an electro-active polymer.

19. The catheter of claim 14 wherein the catheter comprises a flow control valve adapted to control fluid flow between the inlet and the outlet.

20. The shunt system of claim 1 comprising an accelerometer adapted to sense the orientation of the system.

21. A method of operating a catheter comprising controlling if a pressure sensor associated with the catheter is in fluid communication with a fluid in a user's brain or with a fluid in another part of a user's body, bringing the pressure sensor into fluid communication with the fluid which is in the other part of the body, computing a reference level, determining whether an ICP is elevated or depressed by measuring ICP with the pressure sensor, and taking an appropriate action based on whether the ICP is elevated or depressed.

22-61. (canceled)
